(19) United States
(12) Patent Application Publication
(10) Pub. No.: US 2021/0169625 A1
(43) Pub. Date: Jun. 10, 2021

(54) Therapeutic Fluid Dispenser Holder

(71) Applicant: Top Hand Products, LLC, Logan, NM (US)

(72) Inventor: Quinn Morris Courtney, Logan, NM (US)

(21) Appl. No.: 16/949,339

(22) Filed: Oct. 26, 2020

Related U.S. Application Data
(60) Provisional application No. 62/943,899, filed on Dec. 5, 2019.

(51) Int. Cl. A61D 7/00; A01K 13/00; F16M 13/02

(52) U.S. Cl. CPC A61D 7/00; F16M 13/02; A01K 13/003

(57) ABSTRACT

A therapeutic fluid dispenser holder for holding and orienting a bottle for administering fluids to an animal. The therapeutic fluid dispenser holder comprises a sleeve and a hanging line. The sleeve comprises a lower portion having a lower diameter and an upper portion having an upper diameter. The sleeve comprises a sleeve aperture with an outer wall and between an open upper end and an open lower end. The hanging line attaches to a portion of the sleeve and is configured to suspend the therapeutic fluid dispenser holder on an overhead fastener. The outer wall of the sleeve comprises a vertical slot extending from the upper diameter to the lower diameter.

### CROSS-REFERENCE TO RELATED APPLICATIONS

Not applicable.

### STATEMENT REGARDING FEDERALLY SPONSORED RESEARCH OR DEVELOPMENT (IF APPLICABLE)

Not applicable.

### REFERENCE TO SEQUENCE LISTING, A TABLE, OR A COMPUTER PROGRAM LISTING COMPACT DISC APPENDIX (IF APPLICABLE)

Not applicable.

### BACKGROUND OF THE INVENTION

Prior art known to the Applicant includes U.S. Pat. Nos. 5,911,709A, and 3,677,248A.

One feature of a therapeutic fluid dispenser holder 100 can comprise the providing an easy means of swapping out and replacing a bottle 106 during the administration of medicines to an animal 110 by a ranch hand, employee, animal caretaker, and/or vet. For example, in one case, a veterinarian might be positioned proximate to a cattle chute filled with cattle in queue for treatment. The veterinarian can hang said therapeutic fluid dispenser holder 100 on an overhead fastener 226 above the chute, care for each animal in the queue, and rapidly replace said bottle 106 as each is emptied throughout the job.

Existing holders are cumbersome and lack quick replacement of bottles during the job of caring for a plurality of said animal 110. Considering the difficulty of caring for animals and importance of maintaining a calm working environment during the administration of therapeutic fluids, it is surprising that better designs have not yet been presented. Nonetheless, the current system and method of use are intended to advance the art and alleviate pain points in the administration of therapeutic fluids to animals, as well as other uses to which the current system might be put to use by a person skilled in the art.

### BRIEF SUMMARY OF THE INVENTION

A therapeutic fluid dispenser holder for holding and orienting a bottle for administering fluids to an animal. Said therapeutic fluid dispenser holder comprises a sleeve and a hanging line. Said sleeve comprises a lower portion having a lower diameter and an upper portion having an upper diameter. Said sleeve comprises a sleeve aperture with an outer wall and between an open upper end and an open lower end. Said hanging line attaches to a portion of said sleeve and is configured to suspend said therapeutic fluid dispenser holder on an overhead fastener. Said outer wall of said sleeve comprises a vertical slot extending from said upper diameter to said lower diameter. Said hanging line is configured to attach to a hook on a fixed overhead structure, or said overhead fastener. Said sleeve aperture is defined as a space within said sleeve and comprises a substantially conical shape with said upper diameter being larger than said lower diameter. Said sleeve aperture is configured to receive a fluid line of said bottle without requiring a syringe of said bottle to pass through said sleeve aperture by allowing a middle portion of said fluid line to pass through said sleeve aperture at said vertical slot, said open lower end and said open upper end. Said therapeutic fluid dispenser holder further comprises one or more line apertures. Said hanging line is configured to attach to said one or more line apertures. Said one or more line apertures comprise at least a first line aperture, and a second line aperture. Said therapeutic fluid dispenser holder further comprises one or more line mounting extensions. Said one or more line apertures are located in said one or more line mounting extensions. Said one or more line mounting extensions are configured to extend out from said open upper end in a substantially horizontal direction and radially from a center axis of said sleeve.

Said therapeutic fluid dispenser holder for holding and orienting said bottle for administering fluids to said animal. Said therapeutic fluid dispenser holder comprises said sleeve and said hanging line. Said sleeve comprises said lower portion having said lower diameter and said upper portion having said upper diameter. Said sleeve comprises said sleeve aperture with said outer wall and between said open upper end and said open lower end. Said hanging line attaches to a portion of said sleeve and is configured to suspend said therapeutic fluid dispenser holder on said overhead fastener. Said outer wall of said sleeve comprises said vertical slot extending from said upper diameter to said lower diameter.

Said therapeutic fluid dispenser holder for holding and orienting said bottle for administering fluids to said animal. Said therapeutic fluid dispenser holder comprises said sleeve and said hanging line. Said sleeve comprises said lower portion having said lower diameter and said upper portion having said upper diameter. Said sleeve comprises said sleeve aperture with said outer wall and between said open upper end and said open lower end. Said hanging line attaches to a portion of said sleeve and is configured to suspend said therapeutic fluid dispenser holder on said overhead fastener. Said outer wall of said sleeve comprises said vertical slot extending from said upper diameter to said lower diameter. Said hanging line is configured to attach to said hook on said fixed overhead structure, or said overhead fastener. Said sleeve aperture is defined as a space within said sleeve and comprises a substantially conical shape with said upper diameter being larger than said lower diameter. Said sleeve aperture is configured to receive said fluid line of said bottle without requiring said syringe of said bottle to pass through said sleeve aperture by allowing a middle portion of said fluid line to pass through said sleeve aperture at said vertical slot, said open lower end and said open upper end. Said therapeutic fluid dispenser holder is configured to further comprise a plurality of rigidity supports around said outer wall. Said plurality of rigidity supports comprises ribs or raised portions of said sleeve for hindering flexibility in said sleeve and thereby hold said bottle more securely.

### BRIEF DESCRIPTION OF THE SEVERAL VIEWS OF THE DRAWING

FIG. 1 illustrates a perspective overview of a therapeutic fluid dispenser holder 100 in use.

FIG. 2 illustrates a detailed perspective overview of said therapeutic fluid dispenser holder 100, without an animal 110.

FIG. 3 illustrates a perspective overview of said therapeutic fluid dispenser holder 100.

FIGS. 4A and 4B illustrate a perspective overview and lower view of said therapeutic fluid dispenser holder 100.

FIGS. 5A and 5B illustrate an elevated front view and side view of said therapeutic fluid dispenser holder 100.

FIG. 6 illustrates a cross-section perspective overview of said therapeutic fluid dispenser holder 100 with a bottle 106 within a sleeve 102.

FIG. 7 illustrates a cross-section perspective overview of said therapeutic fluid dispenser holder 100.

FIG. 8 illustrates an elevated front view of a hanging line 104.

FIG. 9 illustrates a perspective overview of said sleeve 102.

FIG. 10 illustrates a perspective overview of said therapeutic fluid dispenser holder 100 in a nonpreferred embodiment.

FIG. 11 illustrates a perspective cross-section overview of said therapeutic fluid dispenser holder 100 in a shortened height configuration 1100 with said bottle 106.

FIGS. 12A, and 12B illustrate an elevated side view and front view of said therapeutic fluid dispenser holder 100 having said hanging line 104 with a first s-hook 1200 and a second s-hook 1202 instead of a two carabiners 204.

### DETAILED DESCRIPTION OF THE INVENTION

The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

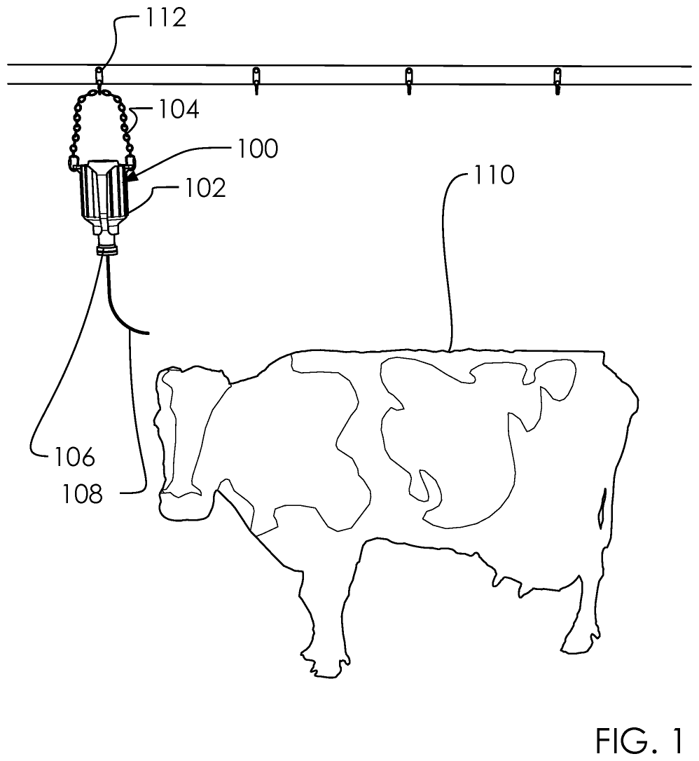

FIG. 1 illustrates a perspective overview of a therapeutic fluid dispenser holder 100 in use.

In one embodiment, said therapeutic fluid dispenser holder 100 can comprise a sleeve 102, and a hanging line 104. In one embodiment, said hanging line 104 can comprise a chain, as illustrated.

Said therapeutic fluid dispenser holder 100 can selectively hold a bottle 106. In one embodiment, said bottle 106 can be held in an inverted position, as illustrated.

One objective of said therapeutic fluid dispenser holder 100 can be to hang said bottle 106 (having a fluid line 108) above an animal 110. One advantage of holding said bottle 106 in an inverted orientation is to ensure that its contents are administered to said animal 110. In one embodiment, said bottle 106 can hold medication, as is known in the art. Exemplary medications can comprise vaccines and/or antibiotics.

In one embodiment, said bottle 106 can comprise a 100 ml-500 ml bottle, as is known in the art, or other sizes according to industry trends. Accordingly, the scale of said therapeutic fluid dispenser holder 100 may change to accommodate different size bottles.

In one embodiment, said sleeve 102 can be made of plastics, metal, PVC, or other suitable materials.

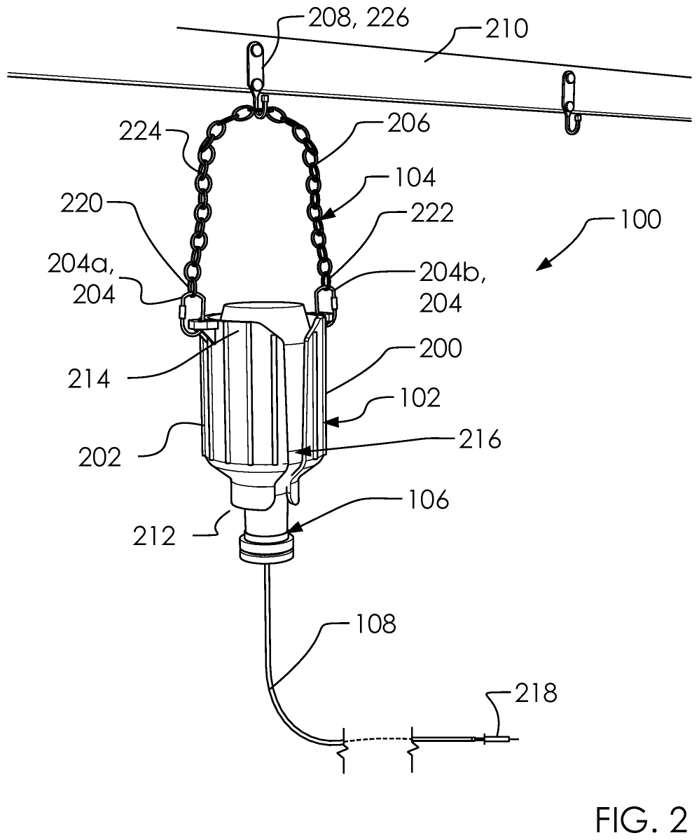

FIG. 2 illustrates a detailed perspective overview of said therapeutic fluid dispenser holder 100, without said animal 110.

In one embodiment, said hanging line 104 can comprise a two carabiners 204 (which can comprise a first carabiner 204a, and a second carabiner 204b), and a connecting line 206 with said two carabiners 204 at each end of said connecting line 206. As illustrated, where said hanging line 104 is a chain, said two carabiners 204 can connect to a first chain link 220 and a last chain link 222, as illustrated, or selectively to one or more intermittent chain links 224 between said first chain link 220 and said last chain link 222, as desired by the user.

In one embodiment, said hanging line 104 can attach to a hook 208 on a fixed overhead structure 210, or an overhead fastener 226, as is known in the art. In one embodiment, said hanging line 104 can comprise chain, bailing wire, or similar hanging materials.

In one embodiment, said sleeve 102 can comprise a lower portion 212, an upper portion 214, and a vertical slot 216.

In one embodiment, said fluid line 108 can attach to a syringe 218 for use in medicating said animal 110. In one embodiment, said syringe 218 can inject medications subcutaneously. The term “therapeutic fluid” in said therapeutic fluid dispenser holder 100 can comprise vaccines, vitamins, antibiotics, or any health-related fluid for injection into an animal.

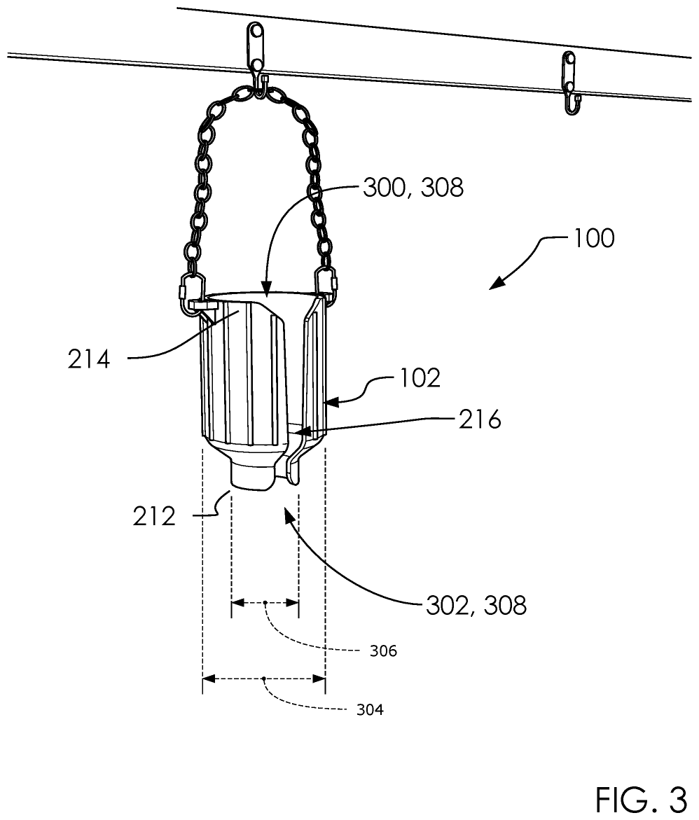

FIG. 3 illustrates a perspective overview of said therapeutic fluid dispenser holder 100.

In one embodiment, said sleeve 102 can comprise an open upper end 300, an open lower end 302, an upper diameter 304, a lower diameter 306, and a sleeve aperture 308.

In one embodiment, said sleeve aperture 308 can be defined as a space within said sleeve 102 and can comprise a substantially conical shape with said upper diameter 304 being larger than said lower diameter 306. In one embodiment, by having a conical shape, said bottle 106 can he held inverted inside of said sleeve 102 without falling through said sleeve aperture 308. Wherein, said bottle 106 can comprise a tapered and more narrow neck proximate to an open end of said bottle 106.

In one embodiment, said fluid line 108 can pass through portions of said sleeve aperture 308 by passing through said vertical slot 216, said open upper end 300 and said open lower end 302 as said bottle 106 is inserted into said sleeve 102.

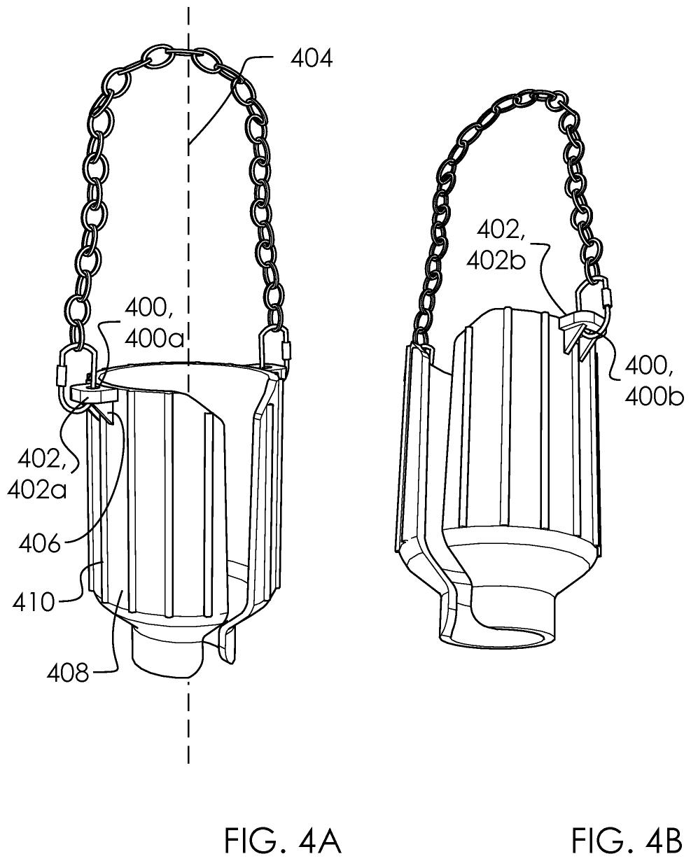

FIGS. 4A and 4B illustrate a perspective overview and lower view of said therapeutic fluid dispenser holder 100.

In one embodiment, said therapeutic fluid dispenser holder 100 can comprise one or more line apertures 400 (which can comprise a first line aperture 400a, and a second line aperture 400b) which can be located in one or more line mounting extensions 402 (which can comprise a first line mounting extension 402a, and a second line mounting extension 402b). In one embodiment, said one or more line apertures 400 can be drilled into sides of said sleeve 102 proximate to said upper portion 214 and/or said open upper end 300 (not illustrated). In one embodiment, said one or more line mounting extensions 402 can extend out from said open upper end 300 in a substantially horizontal direction and radially from a center axis 404 of said sleeve 102. In one embodiment, said one or more line mounting extensions 402 can each be supported by one or more perpendicular supports 406 between an outer wall 408 of said sleeve 102 and a lower portion of said one or more line mounting extensions 402.

In one embodiment, said therapeutic fluid dispenser holder 100 can further comprise a plurality of rigidity supports 410 around said outer wall 408. Said plurality of rigidity supports 410 can extend between said open upper end 300 and said open lower end 302 in a substantially vertical direction. Said plurality of rigidity supports 410 can comprise ribs or raised portions of said sleeve 102 for hindering flexibility in said sleeve 102 and thereby hold said bottle 106 more securely. Said plurality of rigidity supports 410 can be spaced evenly in a radial pattern on said outer wall 408 as rotated around said center axis 404.

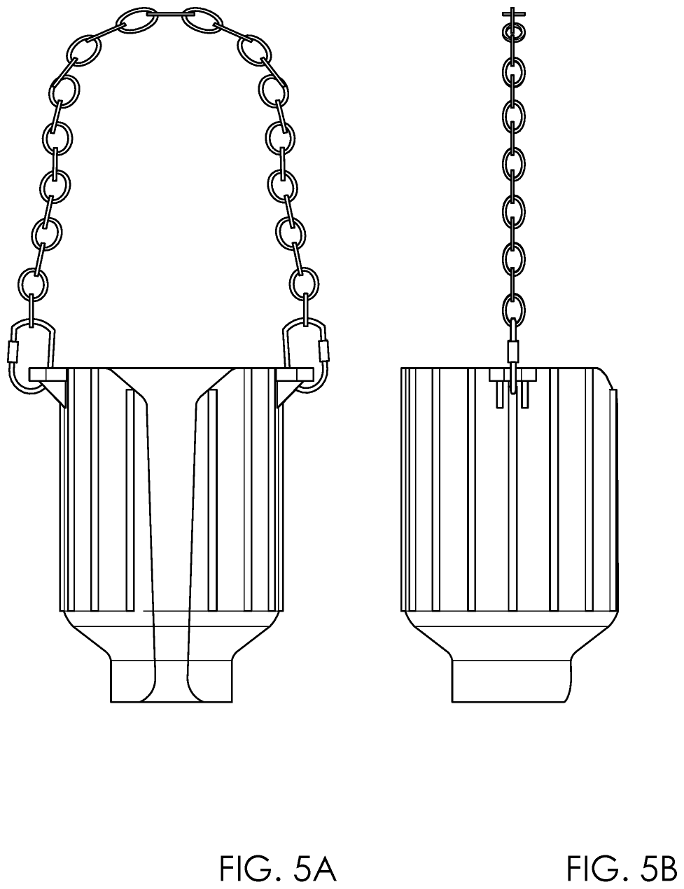

FIGS. 5A and 5B illustrate an elevated front view and side view of said therapeutic fluid dispenser holder 100.

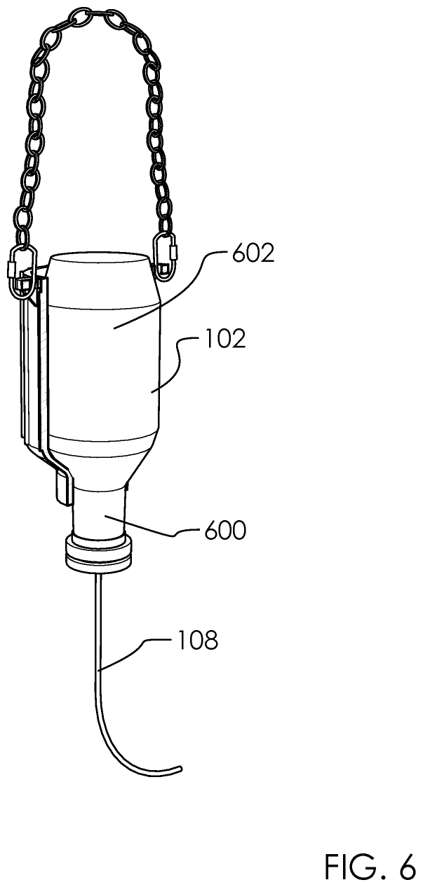

FIG. 6 illustrates a cross-section perspective overview of said therapeutic fluid dispenser holder 100 with said bottle 106 within said sleeve 102.

In one embodiment, said bottle 106 can comprise a neck 600, and a body 602; wherein, said neck 600 can comprise a narrower diameter than said body 602 such that said sleeve 102 can hold said bottle 106 by tapering around said body 602 and allowing said neck 600 to pass through.

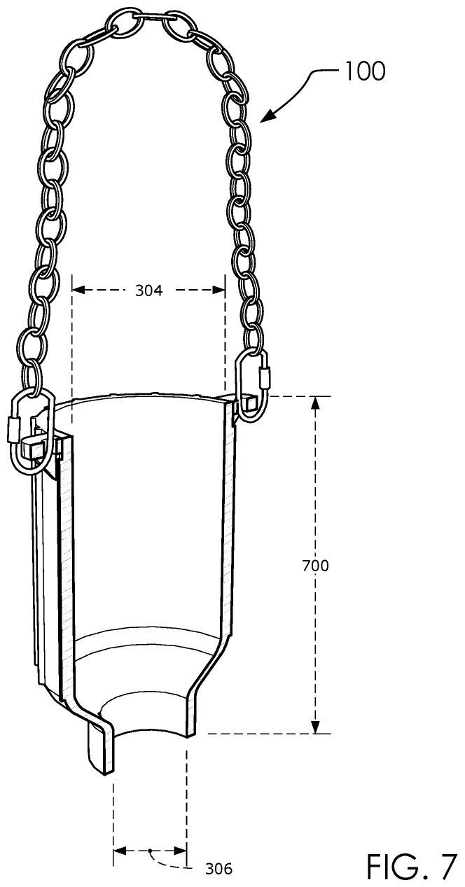

FIG. 7 illustrates a cross-section perspective overview of said therapeutic fluid dispenser holder 100.

In one embodiment, said sleeve 102 can comprise said upper diameter 304, said lower diameter 306 and a height 700.

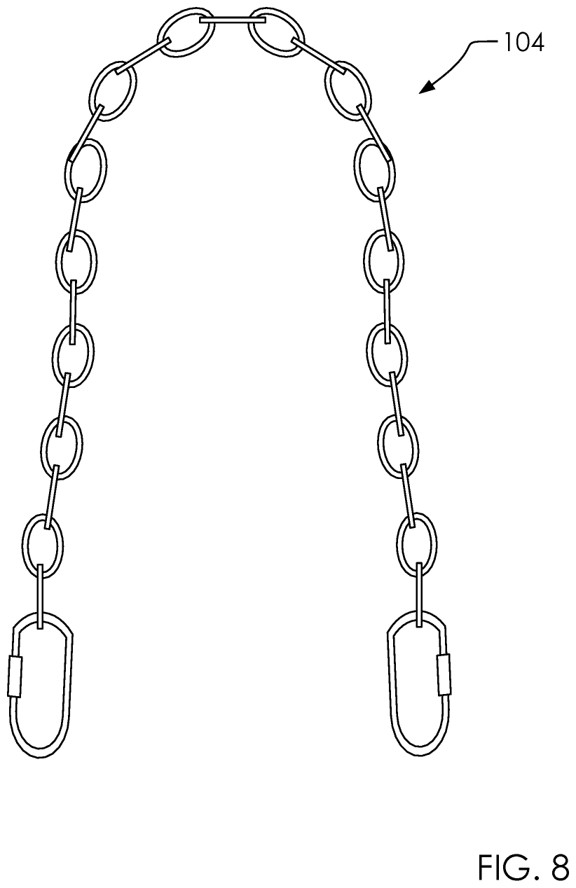

FIG. 8 illustrates an elevated front view of said hanging line 104.

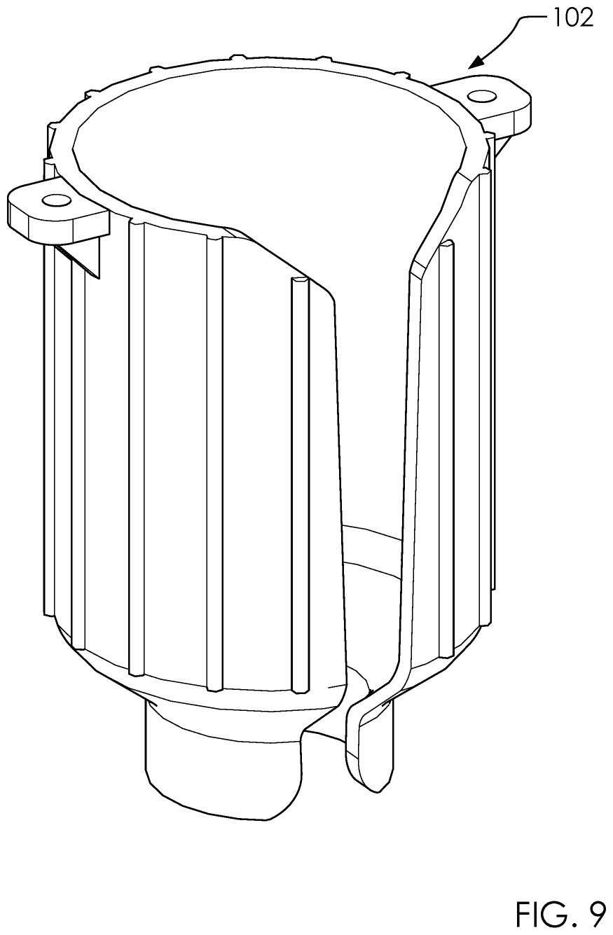

FIG. 9 illustrates a perspective overview of said sleeve 102.

One advantage of having said vertical slot 216 in said sleeve 102 can comprise allowing an end user to remove said fluid line 108 with said bottle 106 without having to thread said syringe 218 through said sleeve aperture 308, avoiding accidental puncturing or harming unintended animals or users while replenishing medications within said therapeutic fluid dispenser holder 100. Therefore, said therapeutic fluid dispenser holder 100 can create a safer work environment.

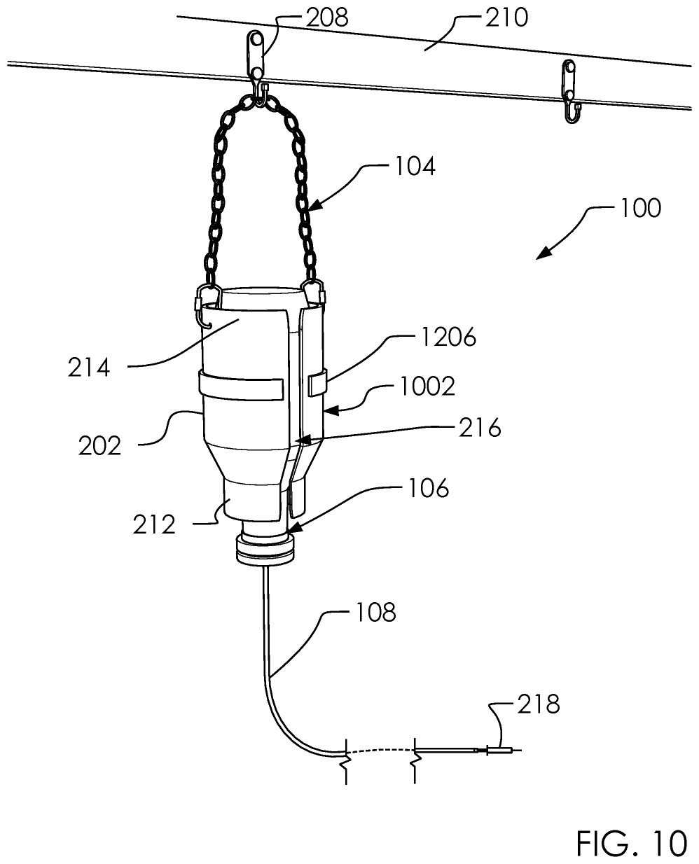

FIG. 10 illustrates a perspective overview of said therapeutic fluid dispenser holder 100 in a nonpreferred embodiment.

In one embodiment, said therapeutic fluid dispenser holder 100 can comprise a solid body 1002, lacking said plurality of rigidity supports 410, but instead uses reinforcement band 1006 to hold said bottle 106 within said therapeutic fluid dispenser holder 100. In one embodiment, said reinforcement band 1006 can wrap around a central portion 202 of said sleeve 102 and provide inward pressure to prevent said sleeve 102 from opening too widely as said bottle 106 presses outward from within said sleeve 102. In one embodiment, where said bottle 106 comprises different external diameters, larger and smaller diameters of said reinforcement band 1006 can be used to sufficiently squeeze around said bottle 106.

In one embodiment, said one or more line apertures 400 are located on said sleeve 102 rather than on said one or more perpendicular supports 406, as discussed above.

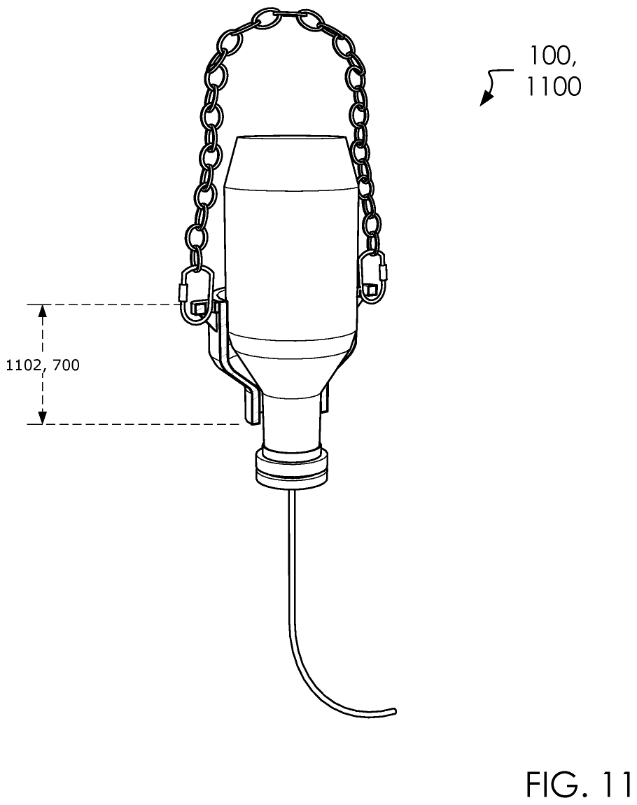

FIG. 11 illustrates a perspective cross-section overview of said therapeutic fluid dispenser holder 100 in a shortened height configuration 1100 with said bottle 106.

In one embodiment, said shortened height configuration 1100 can comprise a lower height 1102 for said height 700. Said shortened height configuration 1100 can comprise said plurality of rigidity supports 410 or not, depending on design preferences.

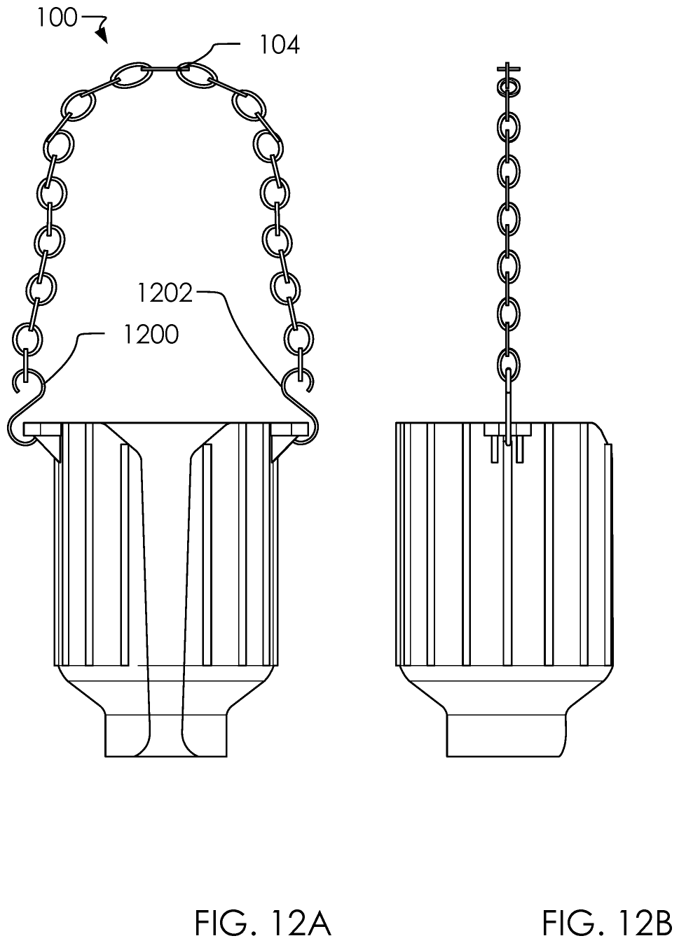

FIGS. 12A, and 12B illustrate an elevated side view and front view of said therapeutic fluid dispenser holder 100 having said hanging line 104 with a first s-hook 1200 and a second s-hook 1202 instead of said two carabiners 204.

The following sentences are presented with reference to the original claims to ensure that all materials presented in the claims are also included in the specification.

Said therapeutic fluid dispenser holder 100 for holding and orienting said bottle 106 for administering fluids to said animal 110. Said therapeutic fluid dispenser holder 100 comprises said sleeve 102 and said hanging line 104. Said sleeve 102 comprises said lower portion 212 having said lower diameter 306 and said upper portion 214 having said upper diameter 304. Said sleeve 102 comprises said sleeve aperture 308 with said outer wall 408 and between said open upper end 300 and said open lower end 302. Said hanging line 104 attaches to a portion of said sleeve 102 and can be configured to suspend said therapeutic fluid dispenser holder 100 on said overhead fastener 226. Said outer wall 408 of said sleeve 102 comprises said vertical slot 216 extending from said upper diameter 304 to said lower diameter 306. Said hanging line 104 can be configured to attach to said hook 208 on said fixed overhead structure 210, or said overhead fastener 226. Said sleeve aperture 308 can be defined as a space within said sleeve 102 and comprises a substantially conical shape with said upper diameter 304 being larger than said lower diameter 306. Said sleeve aperture 308 can be configured to receive said fluid line 108 of said bottle 106 without requiring said syringe 218 of said bottle 106 to pass through said sleeve aperture 308 by allowing a middle portion of said fluid line 108 to pass through said sleeve aperture 308 at said vertical slot 216, said open lower end 302 and said open upper end 300. Said therapeutic fluid dispenser holder 100 further comprises said one or more line apertures 400. Said hanging line 104 can be configured to attach to said one or more line apertures 400. Said one or more line apertures 400 comprise at least said first line aperture 400a, and said second line aperture 400b. Said therapeutic fluid dispenser holder 100 further comprises said one or more line mounting extensions 402. Said one or more line apertures 400 can be located in said one or more line mounting extensions 402. Said one or more line mounting extensions 402 can be configured to extend out from said open upper end 300 in a substantially horizontal direction and radially from said center axis 404 of said sleeve 102.

Said therapeutic fluid dispenser holder 100 for holding and orienting said bottle 106 for administering fluids to said animal 110. Said therapeutic fluid dispenser holder 100 comprises said sleeve 102 and said hanging line 104. Said sleeve 102 comprises said lower portion 212 having said lower diameter 306 and said upper portion 214 having said upper diameter 304. Said sleeve 102 comprises said sleeve aperture 308 with said outer wall 408 and between said open upper end 300 and said open lower end 302. Said hanging line 104 attaches to a portion of said sleeve 102 and can be configured to suspend said therapeutic fluid dispenser holder 100 on said overhead fastener 226. Said outer wall 408 of said sleeve 102 comprises said vertical slot 216 extending from said upper diameter 304 to said lower diameter 306.

Said hanging line 104 comprises a chain comprising said first chain link 220, said last chain link 222 and said one or more intermittent chain links 224.

Said hanging line 104 further comprises said two carabiners 204 comprising said first carabiner 204a and said second carabiner 204b. Said first chain link 220 connects to said first carabiner 204a and said last chain link 222 connects to said second carabiner 204b.

Said sleeve 102 can be made of plastics, metal, PVC, or other suitable materials.

Said hanging line 104 can be configured to attach to said hook 208 on said fixed overhead structure 210, or said overhead fastener 226.

Said sleeve aperture 308 can be defined as a space within said sleeve 102 and comprises a substantially conical shape with said upper diameter 304 being larger than said lower diameter 306.

by having a conical shape, said sleeve 102 can be configured to hold said bottle 106 inverted inside of said sleeve 102 without falling through said sleeve aperture 308. Said bottle 106 comprises a tapered and more narrow neck proximate to an open end of said bottle 106.

Said sleeve aperture 308 can be configured to receive said fluid line 108 of said bottle 106 without requiring said syringe 218 of said bottle 106 to pass through said sleeve aperture 308 by allowing a middle portion of said fluid line 108 to pass through said sleeve aperture 308 at said vertical slot 216, said open lower end 302 and said open upper end 300.

Said therapeutic fluid dispenser holder 100 further comprises said one or more line apertures 400. Said hanging line 104 can be configured to attach to said one or more line apertures 400. Said therapeutic fluid dispenser holder 100 of claim 11. Said one or more line apertures 400 can be drilled into sides of said sleeve 102 proximate to said open upper end 300.

Said therapeutic fluid dispenser holder 100 further comprises said one or more line apertures 400. Said hanging line 104 can be configured to attach to said one or more line apertures 400. Said therapeutic fluid dispenser holder 100 of claim 11. Said one or more line apertures 400 can be drilled into sides of said sleeve 102 proximate to said open upper end 300.

Said one or more line apertures 400 comprise at least said first line aperture 400a, and said second line aperture 400b.

Said therapeutic fluid dispenser holder 100 further comprises said one or more line mounting extensions 402. Said one or more line apertures 400 can be located in said one or more line mounting extensions 402. Said one or more line mounting extensions 402 can be configured to extend out from said open upper end 300 in a substantially horizontal direction and radially from said center axis 404 of said sleeve 102.

Said one or more line mounting extensions 402 can be configured to each be supported by said one or more perpendicular supports 406 between said outer wall 408 of said sleeve 102 and a lower portion of said one or more line mounting extensions 402.

Said one or more line mounting extensions 402 comprise said first line mounting extension 402a, and said second line mounting extension 402b.

Said therapeutic fluid dispenser holder 100 can be configured to further comprise said plurality of rigidity supports 410 around said outer wall 408. Said plurality of rigidity supports 410 comprises ribs or raised portions of said sleeve 102 for hindering flexibility in said sleeve 102 and thereby hold said bottle 106 more securely.

Said plurality of rigidity supports 410 can be configured to extend between said open upper end 300 and said open lower end 302 in a substantially vertical direction.

Said plurality of rigidity supports 410 can be spaced evenly in a radial pattern on said outer wall 408 as rotated around said center axis 404.

Said therapeutic fluid dispenser holder 100 comprises said solid body 1002 and said reinforcement band 1006. Said reinforcement band 1006 can be configured to hold said bottle 106 within said therapeutic fluid dispenser holder 100. Said reinforcement band 1006 can be configured to wrap around said central portion 202 of said sleeve 102 and provide inward pressure to prevent said sleeve 102 from releasing as said bottle 106. where said bottle 106 comprises different external diameters, larger and smaller diameters of said reinforcement band 1006 can be used to sufficiently squeeze around said bottle 106.

These parts are presented in the specification and summarized here for quick reference:
said therapeutic fluid dispenser holder 100,
said sleeve 102,
said hanging line 104,
said bottle 106,
said fluid line 108,
said animal 110,
said central portion 202,
said two carabiners 204,
said first carabiner 204a,
said second carabiner 204b,
said connecting line 206,
said hook 208,
said fixed overhead structure 210,
said lower portion 212,
said upper portion 214,
said vertical slot 216,
said syringe 218,
said first chain link 220,
said last chain link 222,
said one or more intermittent chain links 224,
said overhead fastener 226,
said open upper end 300,
said open lower end 302,
said upper diameter 304,
said lower diameter 306,
said sleeve aperture 308,
said one or more line apertures 400,
said first line aperture 400a,
said second line aperture 400b,
said one or more line mounting extensions 402,
said first line mounting extension 402a,
said second line mounting extension 402b,
said center axis 404,
said one or more perpendicular supports 406,
said outer wall 408,
said plurality of rigidity supports 410,
said neck 600,
said body 602,
said height 700,
said solid body 1002,
said reinforcement band 1006,
said shortened height configuration 1100,
said lower height 1102,
said first s-hook 1200 and
said second s-hook 1202.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A therapeutic fluid dispenser holder for holding and orienting a bottle for administering fluids to an animal, wherein:
said therapeutic fluid dispenser holder comprises a sleeve and a hanging line;
said sleeve comprises a lower portion having a lower diameter and an upper portion having an upper diameter;
said sleeve comprises a sleeve aperture with an outer wall and between an open upper end and an open lower end;
said hanging line attaches to a portion of said sleeve and is configured to suspend said therapeutic fluid dispenser holder on an overhead fastener;
said outer wall of said sleeve comprises a vertical slot extending from said upper diameter to said lower diameter;
said hanging line is configured to attach to a hook on a fixed overhead structure, or said overhead fastener;
said sleeve aperture is defined as a space within said sleeve and comprises a substantially conical shape with said upper diameter being larger than said lower diameter;
said sleeve aperture is configured to receive a fluid line of said bottle without requiring a syringe of said bottle to pass through said sleeve aperture by allowing a middle portion of said fluid line to pass through said sleeve aperture at said vertical slot, said open lower end and said open upper end;
said therapeutic fluid dispenser holder further comprises one or more line apertures;
said hanging line is configured to attach to said one or more line apertures;
said one or more line apertures comprise at least a first line aperture, and a second line aperture;
said therapeutic fluid dispenser holder further comprises one or more line mounting extensions;
said one or more line apertures are located in said one or more line mounting extensions; and
said one or more line mounting extensions are configured to extend out from said open upper end in a substantially horizontal direction and radially from a center axis of said sleeve.

2. A therapeutic fluid dispenser holder for holding and orienting a bottle for administering fluids to an animal, wherein:
said therapeutic fluid dispenser holder comprises a sleeve and a hanging line;
said sleeve comprises a lower portion having a lower diameter and an upper portion having an upper diameter;
said sleeve comprises a sleeve aperture with an outer wall and between an open upper end and an open lower end;
said hanging line attaches to a portion of said sleeve and is configured to suspend said therapeutic fluid dispenser holder on an overhead fastener; and
said outer wall of said sleeve comprises a vertical slot extending from said upper diameter to said lower diameter.

3. The therapeutic fluid dispenser holder of claim 2, wherein:
said hanging line comprises a chain comprising a first chain link, a last chain link and one or more intermittent chain links.

4. The therapeutic fluid dispenser holder of claim 2, wherein:
said hanging line further comprises a first s-hook and a second s-hook; and
said first chain link connects to said first s-hook and said last chain link connects to said second s-hook.

5. The therapeutic fluid dispenser holder of claim 2, wherein:
said hanging line further comprises a two carabiners comprising a first carabiner and a second carabiner; and
said first chain link connects to said first carabiner and said last chain link connects to said second carabiner.

6. The therapeutic fluid dispenser holder of claim 2, wherein:
said sleeve is made of plastics, metal, PVC, or other suitable materials.

7. The therapeutic fluid dispenser holder of claim 2, wherein:
said hanging line is configured to attach to a hook on a fixed overhead structure, or said overhead fastener.

8. The therapeutic fluid dispenser holder of claim 2, wherein:
said sleeve aperture is defined as a space within said sleeve and comprises a substantially conical shape with said upper diameter being larger than said lower diameter.

9. The therapeutic fluid dispenser holder of claim 2, wherein:
by having a conical shape, said sleeve is configured to hold said bottle inverted inside of said sleeve without falling through said sleeve aperture; and
said bottle comprises a tapered and more narrow neck proximate to an open end of said bottle.

10. The therapeutic fluid dispenser holder of claim 2, wherein:
said sleeve aperture is configured to receive a fluid line of said bottle without requiring a syringe of said bottle to pass through said sleeve aperture by allowing a middle portion of said fluid line to pass through said sleeve aperture at said vertical slot, said open lower end and said open upper end.

11. The therapeutic fluid dispenser holder of claim 2, wherein:
said therapeutic fluid dispenser holder further comprises one or more line apertures;
said hanging line is configured to attach to said one or more line apertures;

12. said therapeutic fluid dispenser holder of claim 11, wherein:
said one or more line apertures are drilled into sides of said sleeve proximate to said open upper end.

13. The therapeutic fluid dispenser holder of claim 11, wherein:
said therapeutic fluid dispenser holder further comprises one or more line mounting extensions;
said one or more line apertures are located in said one or more line mounting extensions; and
said one or more line mounting extensions are configured to extend out from said open upper end in a substantially horizontal direction and radially from a center axis of said sleeve.

14. The therapeutic fluid dispenser holder of claim 13, wherein:
said one or more line mounting extensions are configured to each be supported by one or more perpendicular supports between said outer wall of said sleeve and a lower portion of said one or more line mounting extensions.

15. The therapeutic fluid dispenser holder of claim 13, wherein:
said one or more line mounting extensions comprise a first line mounting extension, and a second line mounting extension.

16. The therapeutic fluid dispenser holder of claim 2, wherein:
said therapeutic fluid dispenser holder is configured to further comprise a plurality of rigidity supports around said outer wall; and
said plurality of rigidity supports comprises ribs or raised portions of said sleeve for hindering flexibility in said sleeve and thereby hold said bottle more securely.

17. The therapeutic fluid dispenser holder of claim 16, wherein:
said plurality of rigidity supports are configured to extend between said open upper end and said open lower end in a substantially vertical direction.

18. The therapeutic fluid dispenser holder of claim 16, wherein:
said plurality of rigidity supports are spaced evenly in a radial pattern on said outer wall as rotated around said center axis.

19. The therapeutic fluid dispenser holder of claim 2, wherein:
said therapeutic fluid dispenser holder comprises a solid body and reinforcement band;
said reinforcement band is configured to hold said bottle within said therapeutic fluid dispenser holder;
said reinforcement band is configured to wrap around a central portion of said sleeve and provide inward pressure to prevent said sleeve from releasing as said bottle; and
where said bottle comprises different external diameters, larger and smaller diameters of said reinforcement band is used to sufficiently squeeze around said bottle.

20. A therapeutic fluid dispenser holder for holding and orienting a bottle for administering fluids to an animal, wherein:
said therapeutic fluid dispenser holder comprises a sleeve and a hanging line;
said sleeve comprises a lower portion having a lower diameter and an upper portion having an upper diameter;
said sleeve comprises a sleeve aperture with an outer wall and between an open upper end and an open lower end;
said hanging line attaches to a portion of said sleeve and is configured to suspend said therapeutic fluid dispenser holder on an overhead fastener;
said outer wall of said sleeve comprises a vertical slot extending from said upper diameter to said lower diameter;
said hanging line is configured to attach to a hook on a fixed overhead structure, or said overhead fastener;
said sleeve aperture is defined as a space within said sleeve and comprises a substantially conical shape with said upper diameter being larger than said lower diameter;
said sleeve aperture is configured to receive a fluid line of said bottle without requiring a syringe of said bottle to pass through said sleeve aperture by allowing a middle portion of said fluid line to pass through said sleeve aperture at said vertical slot, said open lower end and said open upper end;
said therapeutic fluid dispenser holder is configured to further comprise a plurality of rigidity supports around said outer wall; and
said plurality of rigidity supports comprises ribs or raised portions of said sleeve for hindering flexibility in said sleeve and thereby hold said bottle more securely.
